SEQUENCE LISTING (2 sequences)

SEQ ID NO: 1 - protein, 175 aa, Artificial Sequence
  synthetic polypeptide
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 17 aa, Artificial Sequence
  synthetic peptide
MTPLGPASSLPQSFLLK

(19) United States
(12) Patent Application Publication
(10) Pub. No.: US 2023/0204597 A1
(43) Pub. Date: Jun. 29, 2023

(54) DETERMINATION OF FREE N-TERMINUS OF PEGFILGRASTIM USING AN ACID PROTEASE

(71) Applicant: AMGEN INC., Thousand Oaks, CA (US)

(72) Inventors: Zhongqi Zhang, Thousand Oaks, CA (US); Bhavana Shah, Thousand Oaks, CA (US)

(21) Appl. No.: 17/910,653

(22) PCT Filed: Mar. 19, 2021
(86) PCT No.: PCT/US21/23100

§ 371 Date: Sep. 9, 2022

Related U.S. Application Data
(60) Provisional application No. 62/992,540, filed on Mar. 20, 2020.

(51) Int. Cl. G01N 33/68; C12Q 1/37

(52) U.S. Cl. CPC G01N 33/6863; C12Q 1/37; G01N 2333/535

(57) ABSTRACT

The present disclosure provides materials and methods for determining the presence of an N-terminal modification on a therapeutic protein, and/or the efficiency of N-terminal modification, such as PEGylation, at the N-terminus of a therapeutic protein such as Filgrastim (wherein the PEGylated version is therefore Pegfilgrastim).

The Sequence Listing, which is a part of the present disclosure, is submitted concurrently with the specification as a text file. The name of the text file containing the Sequence Listing is “55200_Seqlisting.txt”, which was created on Mar. 18, 2021 and is 2,064 bytes in size. The subject matter of the Sequence Listing is incorporated herein in its entirety by reference.

### BACKGROUND

Pegfilgrastim (Neulasta®) is produced by attaching a polyethylene glycol (PEG) polymer to Filgrastim (granulocyte colony stimulating factor; G-CSF, GCSF) using conditions that result in linking on its N-terminal amine through reaction with PEG-aldehyde. However, even using special conditions, a certain percentage of PEG-aldehyde can react with other primary amine groups in Filgrastim. A Filgrastim molecule contains five primary amine groups, the first and most desired is located on the N-terminus, but there are four others on the side chains of lysine residues at site 17, 24, 35 and 41, respectively. To determine that PEGgylation indeed occurs at the N-terminus instead of side chain of lysine residues, the analytical method must be able to distinguish the N-terminus from all lysine residues, among which Lys-17 is most difficult due to its closeness to the N-terminus. It is difficult to separate chromatographically PEGgylated Filgrastim when the PEG is at different sites. Therefore, a fragmentation technique must be applied to cleave the PEGylated Filgrastim into smaller fragments. Due to the large size of PEG (˜20 kDa) and its heterogenous nature, separation of the Filgrastim fragments when PEG is located at different sites remains difficult. For example, to distinguish whether a PEG molecule is attached at the N-terminus (e.g., at the N-terminal methionine, when present) or at, e.g., Lys-17, the two residues must be separated either through chemical or enzymatic methods. While historically Edman degradation has been used to assess N-terminal PEGylation of modified polypeptides, there is a need in the art for additional methods for assessing the efficiency of PEGylation or other conjugations on the N-terminus of Filgrastim.

### SUMMARY OF THE INVENTION

As described herein, the present disclosure provides, in various embodiments, materials and methods for determining the presence of an N-terminal modification on a therapeutic protein, and/or the efficiency of N-terminal modification, such as PEGylation, at the N-terminus of a therapeutic protein such as Filgrastim (wherein the PEGylated version is therefore Pegfilgrastim).

In one embodiment, the present disclosure provides a method of measuring the amount of unmodified (e.g., “free”) N-terminus of a polypeptide, comprising the steps of: (a) incubating a sample comprising the polypeptide with a non-specific protease under conditions that allow cleavage at one or more sites within the polypeptide and only once between N-terminal amino acid position 1 and a first lysine amino acid; (b) separating the cleavage products generated in step (a); and (c) measuring the amount of unmodified, free N-terminus of the polypeptide by comparing to a control standard. In one embodiment, the polypeptide is recombinant.

In one embodiment, the present disclosure provides a method of measuring the amount of unmodified (e.g., “free”) N-terminus of a human granulocyte colony-stimulating factor (G-CSF) polypeptide, comprising the steps of: (a) incubating a sample comprising the G-CSF polypeptide with a non-specific protease under conditions that allow cleavage at one or more sites within the G-CSF polypeptide and only once between N-terminal methionine at position 1 and Lysine at position 16; (b) separating the cleavage products generated in step (a); and (c) measuring the amount of unmodified, free N-terminus of the G-CSF polypeptide by comparing to a control standard. In one embodiment, the G-CSF polypeptide is recombinant.

In other embodiments, an aforementioned method is provided wherein said sample comprises a mixture of modified G-CSF polypeptide and unmodified G-CSF polypeptide, and wherein the modified G-CSF polypeptide comprises at least one polyethylene glycol (PEG) modification.

In still another embodiment, an aforementioned method is provided wherein the G-CSF polypeptide is selected from the group consisting of Pegfilgrastim (Neulasta®), Pegfilgrastim-jmdb (Fulphila®), INN-Pegfilgrastim (Pelgraz®), Lapelga®, Pelmeg®, Pegfilgrastim-cbqv (Udenyca®), Pegfilgrastim-bmez (Ziextenzo®), and Grasustek®. In one embodiment, the G-CSF polypeptide is Pegfilgrastim (Neulasta®).

The present disclosure also provides, in various embodiments, an aforementioned method wherein the non-specific protease cleaves between leucine at position 15 and leucine at position 16 and produces a peptide of 15 amino acids in length (peptide M1-L15).

In other embodiments, an aforementioned method is provided wherein the non-specific protease is pepsin.

The present disclosure provides, in various embodiments, an aforementioned method wherein the conditions in step (a) comprise incubating (a) at a pH of about 1.5 to about 4.0, (b) at a temperature of about 25° C. to about 60° C., and (c) for a time of about 5 minutes to about 60 minutes. In one embodiment, the conditions comprise incubating (a) at a pH of about 2.2, (b) at a temperature of about 37° C., and (c) for a time of about 15 minutes.

In still other embodiments, an aforementioned method is provided wherein the separation of step (b) is carried out under conditions that allow separation of peptide M1-L15 from other cleavage products. In one embodiment, the separation of step (b) is carried out by a method selected from chromatography and electrophoresis. In another embodiment, the chromatography is selected from the group consisting of high-performance liquid chromatography (HPLC) and ultrahigh-performance liquid chromatography (UHPLC). In another embodiment, the HPLC is reversed phase HPLC (RP-HPLC). In still other embodiments, the chromatography comprises a column and trifluoroacetic acid (TFA) at a concentration of about 0.01% v/v to about 0.2% v/v. In a related embodiment, the TFA concentration is about 0.02% v/v to about 0.03% v/v. In yet another embodiment, the TFA concentration is about 0.025% v/v.

In other embodiments, an aforementioned method is provided wherein the measuring step (c) is carried out by mass spectrometry. In one embodiment, the mass spectrometry is selected from electrospray MS and Matrix-assisted laser desorption ionization-time of flight mass spectrometry (MALDI-TOF MS).

In still other embodiments, an aforementioned method is provided wherein the control standard comprises a known amount of modified G-CSF polypeptide and a known amount of unmodified G-CSF polypeptide.

The present disclosure provides, in one embodiment, a method of measuring the amount of unPEGylated, free N-terminus of Pegfilgrastim (Neulasta®) comprising the steps of: (a) incubating a sample comprising Pegfilgrastim (Neulasta®) with a non-specific protease under conditions that allow cleavage at one or more sites within the Pegfilgrastim (Neulasta®) and only once between N-terminal methionine at position 1 and Lysine at position 16, and wherein said conditions comprise incubating (a) at a pH of about 2.2, (b) at a temperature of about 37° C., and (c) for a time of about 15 minutes; (b) separating the cleavage products generated in step (a) by reversed phase HPLC (RP-HPLC); and (c) measuring the amount of unPEGylated, free N-terminus of Pegfilgrastim (Neulasta®) by comparing to a control standard.

### BRIEF DESCRIPTION OF THE DRAWINGS

FIG. 1 shows the sequence (SEQ ID NO: 1) of Pegfilgrastim showing the potential PEGylation sites.

FIG. 2 shows chymotrypsin digests comparison for Filgrastim and Pegfilgrastim

FIG. 3 shows the peptide mapping profile of Filgrastim showing pepsin digestion that generated a major N-terminal peptide M1-L15. Chromatogram was obtained on an Agilent 1260 system with an acetonitrile gradient of 2% to 35% acetonitrile in 30 minutes. Mobile phase contained 0.02% (v/v) TFA

FIG. 4 shows a comparison of peptide map profiles of Filgrastim and Pegfilgrastim digested with pepsin.

FIG. 5A shows the overlay profiles of Pegfilgrastim and Pegfilgrastim spiked with 5% Filgrastim with 0.02% (v/v) TFA eluent with six different UPLC column lots. FIG. 5B shows the overlay profiles of Pegfilgrastim and Pegfilgrastim spiked with 5% Filgrastim with 0.025% (v/v) TFA eluent with six different UPLC column lots. FIG. 5C shows the overlay profiles of Pegfilgrastim (black trace) and Pegfilgrastim spiked with 5% Filgrastim (blue trace) with 0.03% (v/v) TFA eluent with four different UPLC column lots.

FIG. 6 shows the profile for Pegfilgrastim reference standard spiked with 5% Filgrastim reference standard with 0.025% (v/v) TFA in the eluent.

FIG. 7 shows a chromatogram comparison of pepsin digest of Pegfilgrastim, Neupogen, Epogen and Romiplostim.

FIG. 8 shows the plot for determined %free N-terminal methionine for each spiked level.

FIG. 9 shows the residual plot for % free N-terminal methionine determined at each level.

FIG. 10 shows the stability comparison for Level 3 (˜2% free N-terminal methionine) sample chromatogram.

FIG. 11A shows Robustness for Total Area. FIG. 11B shows Robustness for Noise (p to p). FIG. 11C shows Robustness for P2 RT. FIG. 11D shows Robustness for P2 Area. FIG. 11E shows Robustness for P3 RT. FIG. 11F shows Robustness for P3 Area. FIG. 11G shows Robustness for P1 RT.

FIG. 12 shows robustness of % Free N-terminal methionine determination.

### DETAILED DESCRIPTION

The present disclosure addresses the aforementioned need in the art by providing methods and materials useful for determining the presence of an N-terminal modification on a therapeutic protein, and/or the efficiency of N-terminal modification, such as, in one embodiment, PEGylation, at the N-terminus of a therapeutic protein such as Filgrastim (and wherein the PEGylated version is therefore Pegfilgrastim).

In various embodiments, Pegfilgrastim is digested with pepsin, a nonspecific protease, under acidic condition. Digested peptides are separated by reversed phase high-performance liquid chromatography (RP-HPLC) with ultraviolet (UV) detection. The proteolytic peptide containing the N-terminal 15 residues is used for quantitation of free N-terminal methionine with a standard addition method by spiking a known amount of Filgrastim in the Pegfilgrastim sample. Pepsin peptide map profile of sample is compared with Pegfilgrastim reference standard to confirm identity.

Definitions

As used herein, “Filgrastim” refers to Filgrastim (Neupogen®) and can be used interchangeably with “G-CSF.” Biosimilars that are also contemplated by the present disclosure include, but are not limited to, Filgrastim-aafi (Nivestym®), tbo-filgrastim (Granix®), Filgrastim-sndz (Zarxio®).

As used herein “Pegfilgrastim” refers to Pegfilgrastim (Neulasta®) and is a PEGylated version of Filgrastim. Biosimilars that are also contemplated by the present disclosure include, but are not limited to, Pegfilgrastim-jmdb (Fulphila®), INN-Pegfilgrastim (Pelgraz®), Lapelga®, Pelmeg®, Pegfilgrastim-cbqv (Udenyca®), Pegfilgrastim-bmez (Ziextenzo®), and Grasustek®.

As used herein, the term “G-CSF” means “granulocyte colony-stimulating growth factor.” As used herein, G-CSF can be chemically or genetically modified and produced recombinantly by methods known in the art. G-CSF can be modified, e.g., with PEG (Filgrastim) or other molecules. In one embodiment, the G-CSF is modified at the N-terminus. In another embodiment, the G-CSF is modified at the N-terminal methionine. Unless otherwise noted, the term G-CSF refers to Filgrastim and the term PEGylated G-CSF or G-CSF conjugate refers to Pegfilgrastim.

The phrase “at least 1” as used herein can be 1, 2, 3, 4, 5, 6. 7, 8, 9, 10 or more.

“Polyethylene glycol” or “PEG” is a polyether compound with many applications, from industrial manufacturing to medicine. PEG is also known as polyethylene oxide (PEO) or polyoxyethylene (POE), depending on its molecular weight. The structure of PEG is commonly expressed as H—(O—CH2-CH2)n-OH.

As used herein, the term “non-specific protease” means an enzyme that catalyzes proteolysis, the breakdown of proteins into smaller polypeptides or single amino acids, without a strict requirement amino acid sequence substrate. Exemplary non-specific proteases without strict substrate requirements contemplated herein include pepsin (and its precursor pepsinogen), chymotrypsin, elastase, papain, protease type XIII, and thermolysine,

As used herein, the terms “protein” and “polypeptide” are used interchangeably and mean any chain of at least five naturally or non-naturally occurring amino acids linked by peptide bonds. As used herein, the terms “isolated” and “purify” are used interchangeably and mean to reduce by 1%, 2%, 3%, 4%, 5%, 10%, 15%, 20%, 25%, 30%, 35%, 40%, 45%, 50%, 55%, 60%, 65%, 70%, 75%, 80%, 85%, 90% or 95%, or more, the amount of heterogenous elements, for example biological macromolecules such as proteins or DNA, that may be present in a sample comprising a protein of interest. The presence of heterogenous proteins can be assayed by any appropriate method including High-performance Liquid Chromatography (HPLC), gel electrophoresis and staining and/or ELISA assay.

Methods of Measuring Unmodified Polypeptides

As described herein, the present disclosure provides in one embodiment a method of measuring the amount of unmodified, free N-terminus of a human granulocyte colony-stimulating factor (G-CSF) polypeptide, comprising the steps of (a) incubating a sample comprising the G-CSF polypeptide with a non-specific protease under conditions that allow cleavage at one or more sites within the G-CSF polypeptide and only once between N-terminal methionine at position 1 and Lysine at position 17; (b) separating the cleavage products generated in step (a); and (c) measuring the amount of unmodified, free N-terminus of the G-CSF polypeptide by comparing to a control standard.

In various other embodiments, the methods described herein are useful for measuring the amount of free N-terminus for any polypeptide, including recombinant therapeutic polypeptides such as antibodies and the like.

PEGylation is being used as a universal therapeutic technique to provide diverse conjugation with aptamers, enzymes, proteins, low molecular-weight drugs, and antibodies, and has expanded clinical applications for biopharma industries. PEGylation is a process through which polyethylene glycol (PEG) chains are conjugated to proteins (therapeutic proteins), peptides, or any molecule. Through the PEGylation process, the molecular mass of the therapeutic protein is increased and can (thus) guard the therapeutic protein from proteolytic enzymes and degradation improve pharmacokinetics.

In one embodiment of the present disclosure, the efficiency of N-terminal PEGylation is determined for Filgrastim. In other embodiments, various other N-terminal modifications (other than PEGylation) are contemplated, including but limited to polysaccharides such as dextran and heparosan. Besides PEG, other polymeric moieties are useful conjugation partners with G-CSF. For example, WO 02/09766 discloses, inter alia, biocompatible protein-polymer compounds produced by conjugation of biologically active protein with a biocompatible polymer derivative. The biocompatible polymer is a highly reactive branched polymer, and the resulting conjugates contain a long linker between the polymer and polypeptide. Examples of biocompatible polymers according to WO 02/09766 are PEG, PPG, polyoxyethylene (POE), polytrimethylene glycol, polylactic acid and its derivatives, polyacrylic acid and its derivatives, polyamino acids, polyurethane, polyphosphazene, poly(L-lysine), polyalkylene oxide (PAO), water-soluble polymers such as polysaccharide, dextran, and non-immunogenic polymers such as polyvinyl alcohol and polyacryl amide.

WO 96/11953 describes N-terminally chemically modified protein compounds and methods for their production. Specifically, G-CSF compositions are described which result from coupling a water-soluble polymer to the N-terminus of G-CSF. Examples of water-soluble polymers listed in WO 96/11953 are copolymers of ethylene glycol and propylene glycol, carboxymethyl cellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl pyrrolidone)polyethylene glycol, PPG homopolymers, polypropylene oxide/ethylene oxide copolymers or polyoxyethylated polyols. Other modifications are described in U.S. Pat. No. 8,207,112, incorporated by reference in its entirety herein.

As described in U.S. Pat. No. 5,824,784, incorporated by reference in its entirety herein, methods for both N-terminally modified G-CSF (e.g., Filgrastim) as well as reductive alkylation methods (which exploits differential reactivity of different types of primary amino groups (lysine versus the N-terminal) available for derivatization) provide for a substantially homogenous mixture of monopolymer/protein conjugate. “Substantially homogenous” as used herein means that the only polymer/protein conjugate molecules observed are those having one polymer moiety. The preparation may contain unreacted (i.e., lacking polymer moiety) protein. As ascertained by peptide mapping and N-terminal sequencing and as described in U.S. Pat. No. 5,824,784, one example provides for a preparation which is at least 90% monopolymer/protein conjugate, and at most 10% unreacted protein. Preferably, the N-terminally mono-PEGylated material is at least 95% of the preparation (as in the working example below) and most preferably, the N-terminally mono-PEGylated material is 99% of the preparation or more. The monopolymer/protein conjugate has biological activity. The present “substantially homogenous” N-terminally PEGylated G-CSF preparations provided herein are those which are homogenous enough to display the advantages of a homogenous preparation, e.g., ease in clinical application in predictability of lot to lot pharmacokinetics.

Chemotherapy-induced neutropenia (CIN) is a common and serious complication of myelosuppressive chemotherapy. It is associated with significant morbidity and mortality and can increase the cost of cancer therapy. In these cases, colony stimulating factor is necessary to restore important cells for immune function. For over twenty years, granulocyte colony-stimulating factors (G-CSFs; Filgrastims) have been a pillar of treatment and prevention of CIN, and have been found to reduce the risk of neutropenia across various patient settings, decrease the incidence of febrile neutropenia, reduce the incidence of infection, reduce the requirement for treatment with antibiotics, and accelerate neutrophil recovery.

Filgrastim is a recombinant, non-pegylated human granulocyte colony stimulating factor (G-CSF) analog. It is marketed as the brand name Neupogen® by Amgen (initially approved in 1998) and as Nivestym®, a biosimilar agent by Pfizer. Neupogen®/filgrastim has been approved for various indications. Tbo-filgrastim, which is marketed by Sicor Biotech and FDA approved on Aug. 29, 2012, contains the same active ingredient as Neupogen® and is biologically similar, but it is formulated to be short-acting. The FDA also approved the biosimilar Zarxio® (filgrastim-sndz) and is indicated for use in the same conditions as Neupogen. Zarxio® is marketed by Sandoz.

Pegfilgrastim is a PEGylated form of the recombinant human granulocyte colony-stimulating factor (G-CSF) analogue, Filgrastim. It is used, among other reasons, to decrease the incidence of infection, as manifested by febrile neutropenia, in patients with non-myeloid cancer receiving myelosuppressive anti-cancer treatment. Due to the relatively short circulating half-life of Filgrastim, a 20 kDa PEG moiety was covalently conjugated to the N-terminus of Filgrastim (at the methionine residue) to develop a longer acting version of the drug. Due to a longer half-life and slower elimination rate than Filgrastim, Pegfilgrastim requires less frequent dosing than Filgrastim. However, Pegfilgrastim retains the same biological activity as Filgrastim and binds to the same G-CSF receptor to stimulate the proliferation, differentiation, and activation of neutrophils.

First developed by Amgen, Pegfilgrastim was initially approved by the FDA in 2002 and marketed as Neulasta®. There are several Pegfilgrastim biosimilars (Fulphila®, Pelgraz® or Lapelga®, Pelmeg®, Udenyca®, Ziextenzo®, and Grasustek®) that are approved for the same therapeutic indication by Health Canada, European Union (EU), and FDA.

The amino acid sequence of Filgrastim and Pegfilgrastim is as follows:

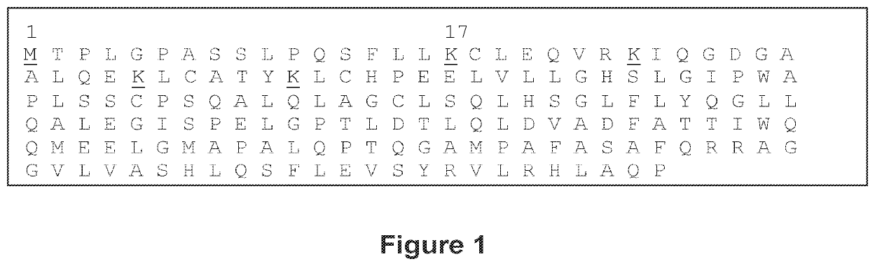

(SEQ ID NO: 1)
MTPLGPASSLPQSFLLKCLEQVRKIQGDGAALQEKLCATYKLCHPEELVL
LGHSLGIPWAPLSSCPSQALQLAGCLSQLHSGLFLYQGLLQALEGISPEL
GPTLDTLQLDVADFATTIWQQMEELGMAPALQPTQGAMPAFASAFQRRAG
GVLVASHLQSFLEVSYRVLRHLAQP (FIG. 1).

The N-terminal 17 residues of Filgrastim is MTPLGPASSLPQSFLLK (SEQ ID NO: 2). None of these residues can be cleaved readily by commonly used proteases such as trypsin (cuts after K and R), Lys-C (cuts after K), Glu-C (cuts after E), Asp-N (cuts before D), and Arg-C (cuts after R). As a result, Edman degradation (performed on an automated N-terminal sequencer) was historically used as a release assay of Pegfilgrastim to cleave residues one-by-one from the N-terminus. Un-PEGgylated free N-terminus was determined by the recovered methionine residue in the first Edman degradation cycle.

The present disclosure provides, in various embodiments, the first use of nonspecific proteases to cleave the N-terminal residues, wherein a single clean cut between the N-terminal methionine and Lys-17 is produced. Because pepsin is a non-specific protease, it can potentially cut at various sites between the two residues. As described herein, conditions have been identified that generate a clean cut between residues Leu-15 and Leu-16 of SEQ ID NO: 2. Additionally, because pepsin works at acidic condition at which the protein is denatured, no reduction/alkylation is required, making sample preparation much more convenient. The resulting proteolytic peptides are separated, in certain embodiments, by reversed-phase HPLC and monitored by UV absorbance. The N-terminal peptide, referred to herein as “M1-L15” is well resolved from other peaks and is used for accurately and reproducibly quantifying the free N-terminus. The late eluting PEGgylated N-terminal peptide can be used for identification purpose.

As is known in the art, pepsin is an endopeptidase that breaks down proteins or polypeptides into smaller peptides or amino acids. It is produced in the chief cells of the stomach lining and is one of the main digestive enzymes in the digestive systems of humans and many other animals, where it helps digest the proteins in food. Pepsin is an aspartic protease, using a catalytic aspartate in its active site. It is one of two principal proteases in the human digestive system, the other two being chymotrypsin and trypsin. During the process of digestion, these enzymes, each of which is specialized in severing links between particular types of amino acids, collaborate to break down dietary proteins into their components, i.e., peptides and amino acids, which can be readily absorbed by the small intestine. Pepsin is most efficient in cleaving peptide bonds between hydrophobic amino acids such as phenylalanine, tryptophan, tyrosine, and leucine. Pepsin's proenzyme, pepsinogen, is released by the chief cells in the stomach wall, and upon mixing with the hydrochloric acid of the gastric juice, pepsinogen activates to become pepsin.

Separation of the digestion products can be accomplished in many ways, according to the present disclosure. By way of example, chromatography and electrophoresis are contemplated according to some embodiments. For example, high-performance liquid chromatography (HPLC), ultrahigh-performance liquid chromatography (UHPLC), reversed phase HPLC (RP-HPLC), hydrophilic interaction chromatography (HILIC), and ion-exchange chromatography.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### EXAMPLE 1

### Pepsin Digestion and Separation of Products

A. Protease Selection

FIG. 1 shows the sequence of Pegfilgrastim, with potential PEGylation sites highlighted in red. In order to distinguish N-terminus from lysine-17, a cleavage must happen between Met-1 and Lys-17. Because there is no cleavage site for common specific protease, nonspecific proteases such as pepsin or chymotrypsin were used.

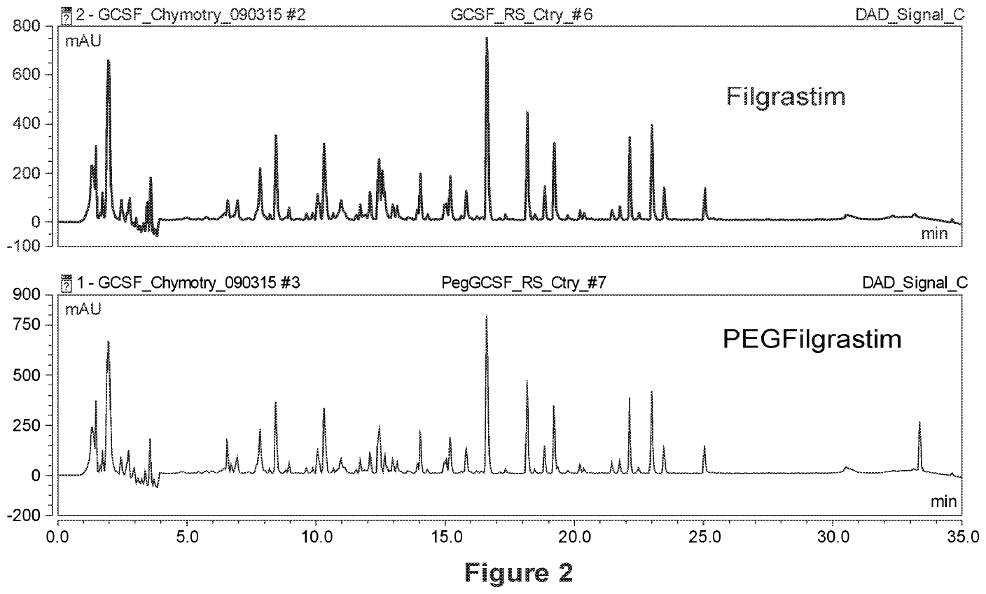

Assessment of pepsin and chymotrypsin showed pepsin is more promising than chymotrypsin. As seen in FIG. 2, chymotrypsin digests of Filgrastim and Pegfilgrastim did not show well-resolved free N-terminal methionine peak from other peptide peaks. Additionally, because pepsin works at acidic pH when the protein is denatured, no reduction/alkylation is required, making sample preparation much easier and straightforward.

B. Pepsin Digestion of Filgrastim

To perform pepsin digestion of Filgrastim or Pegfilgrastim, 120 μL of protein sample at 1 mg/mL was used, 60 μL of 0.3M phosphate buffer at pH 2.2 and add 10 μL of pepsin solution at 0.48 mg/mL was added, followed by incubating at 50° C. for 5 minutes or 37° C. for 15 minutes. After incubation, the digestion was quenched by adding 10 μL of 1N NaOH.

Digestion conditions were tested on Filgrastim reference standard (RS) to maximize the free N-terminal peptide. The digestion conditions were optimized at either 50° C. for 5 minutes or 37° C. for 15 minutes, which gave similar results. A final condition of 37° C. for 15 minutes was selected because it is likely to have lower relative error associated with the longer digestion time, and result in a more robust condition.

In order to see how the N-terminal region is digested by pepsin, Filgrastim (RS) was digested and analyzed on an Agilent 1290 HPLC system using a Waters CSH 100×2.1 mm column at 50° C., eluted at 0.2 mL/min with an acetonitrile gradient containing 0.02% (v/v) TFA in each mobile phase. The HPLC was directly connected to a Thermo Scientific LTQ-Orbitrap system to collect mass and MS/MS data for identification of each eluted peptide.

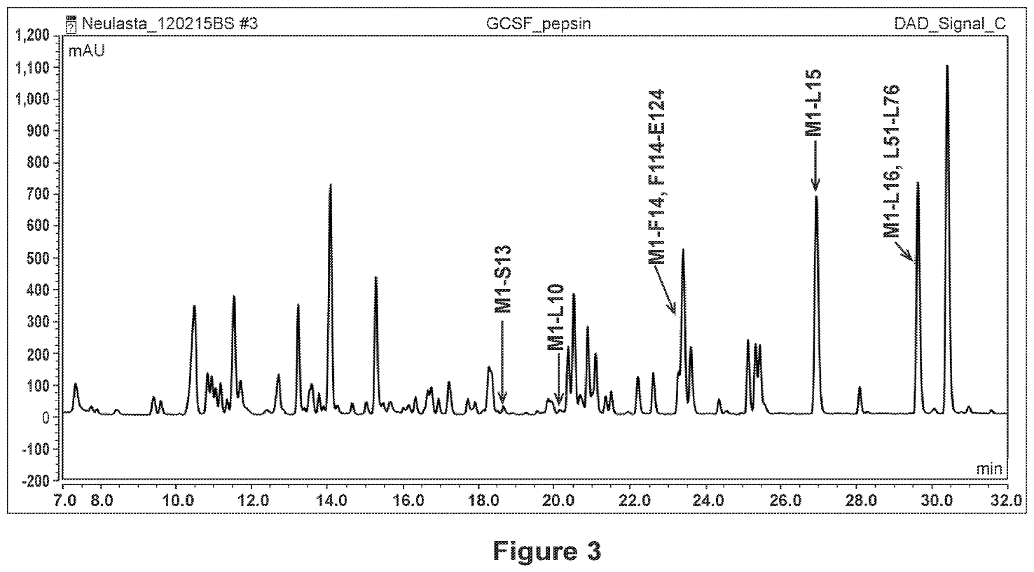

FIG. 3 shows the peptide map profile of Filgrastim RS digested with pepsin. Pepsin digestion of Filgrastim generated a major N-terminal peptide M1-L15. This peptide was well resolved without any interference from other peptides in the chromatogram, suggesting the possibility of using this peptide to quantify free N-terminal methionine. Other N-terminal peptides, such as M1-L10, M1-S13, M1-F14 (co-eluting with peptide F114-E124) and M1-L16 (co-eluting with peptide L51-L76) are also observed, but they all have low abundance relative to the M1-L15 peptide as seen in mass spec intensity of each peptide. These low abundance four peptides were not considered in the quantitation of free N-terminal methionine as they should not have significant contribution to the quantitation accuracy.

C. Pepsin Digestion of Pegfilgrastim

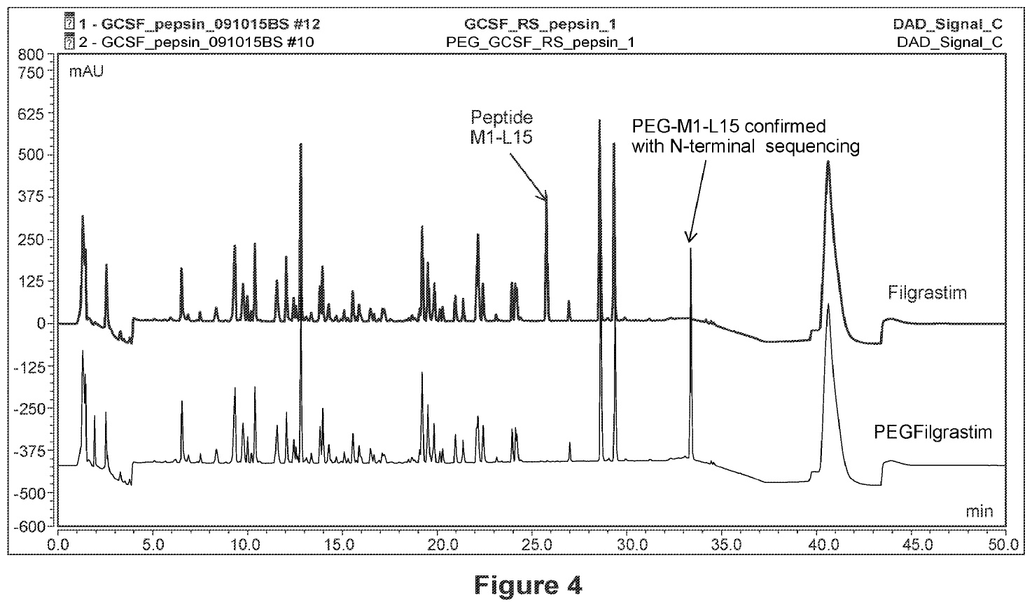

A Pegfilgrastim sample was digested by pepsin at 37° C. for 15 minutes and the peptide map profiles were compared to that of Filgrastim (FIG. 4). Peptides were identified by online mass spectrometric detection. Except for the disappearance of the N-terminal free peptide (M1-L15) and the appearance of PEGylated N-terminal peptide (PEG-M1-L15), very similar profiles were obtained, indicating that the presence of PEGylation does not affect pepsin digestion. No distinct mass can be determined for the extra late eluting peak in Pegfilgrastim due to the heterogeneous nature of PEGylation. This peak was isolated and analyzed by MALDI-TOF MS as well as N-terminal sequencing. MALDI-TOF gave a broad peak with mass ˜23000Da and N-terminal sequencing confirmed its sequence as the N-terminal M1-L15, where in the first cycle, instead of methionine, threonine (actual 2nd residue) was observed.

D. Robustness of Pepsin Digestion

Due to the nonspecific nature of pepsin, a concern is that pepsin material obtained from different sources or vendor lots may exhibit different activity and therefore generating different peptide map profiles. To test the robustness of pepsin digestion, pepsin material obtained from six different sources were used to digest a Filgrastim sample (lot 1039502) and the resulting chromatograms were compared. Description of the six pepsin materials are shown in Table 1:

TABLE 1
Description of the six pepsin materials used for robustness evaluation.

| | | | | Activity |
|---|---|---|---|---|
| Material | Vendor | Part # | Lot # | (unit/mg) |
| 1 | Sigma | P6887 | 074K7717 | 3200-4500 |
| 2 | Sigma | P6887 | SLBM3033V | 3200-4500 |
| 3 | Sigma | P7012 | SLBL6640V | >2500 |
| 4 | Sigma | P7125 | SLBN1170V | >450 |
| 5 | Roche | 10108057001 | 70444420 | ~2500 |
| 6 | Affimetrix | 20010 | 4184818 | >2500 |

The chromatograms showed that the digestion using pepsin is robust and reproducible, regardless of the source of pepsin, as long as the pepsin activity is no less than 2500 unit/mg. MS intensities of different N-terminal peptides generated in this set of data demonstrate the reproducibility of the digest and the dominance of M1-L15 when different sources of pepsin are used.

E. Chromatographic Separation

To quantify free N-terminal methionine in Pegfilgrastim using the N-terminal peptide Ml-L15, the peptide must be separated from all other nearby eluting peptides. A sample containing 95% Pegfilgrastim reference standard (RS) and 5% Filgrastim RS was prepared and used for optimizing chromatography separation. A Waters CSH C18 column (2.1×100 mm. 1.7 μm) was used. After some gradient optimization, the following chromatographic conditions were selected.

Mobile phase A: 0.025% TFA in water

Mobile phase B: 0.025% TFA in acetonitrile

Column temperature: 50° C.

Detection wavelength: 214 nm

Flow rate: 0.2 mL/min

Gradient:

| Time(min) | % B |
|---|---|
| 0.0-1.0 | 2.0 |
| 20.0 | 25.0 |
| 30.0 | 30.0 |
| 40.0-42.0 | 99.0 |
| 43.0-55.0 | 2.0 |

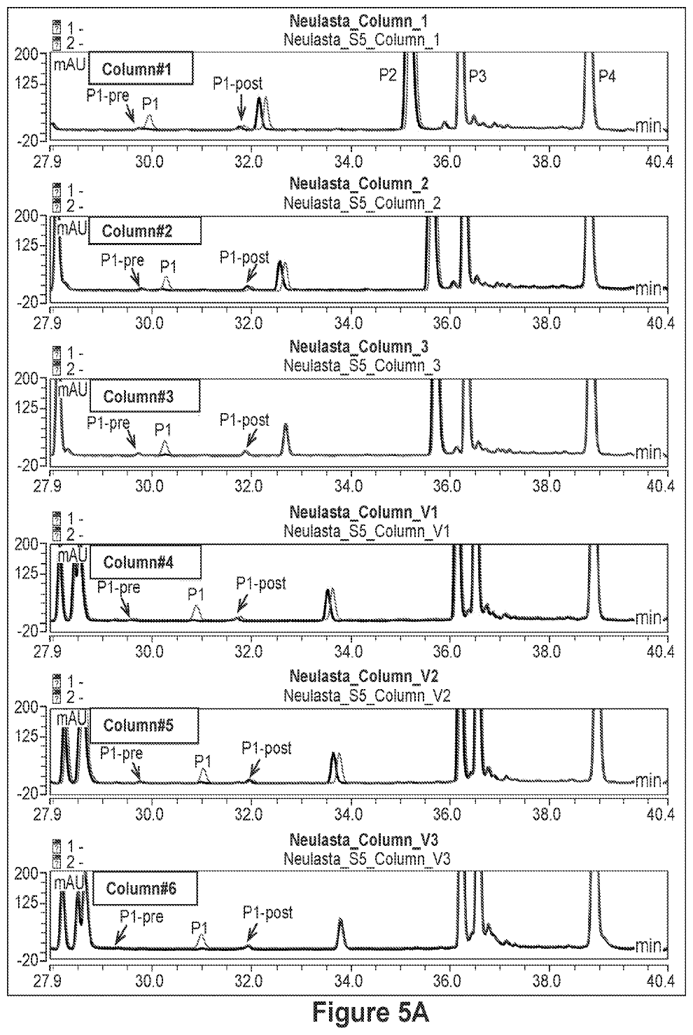

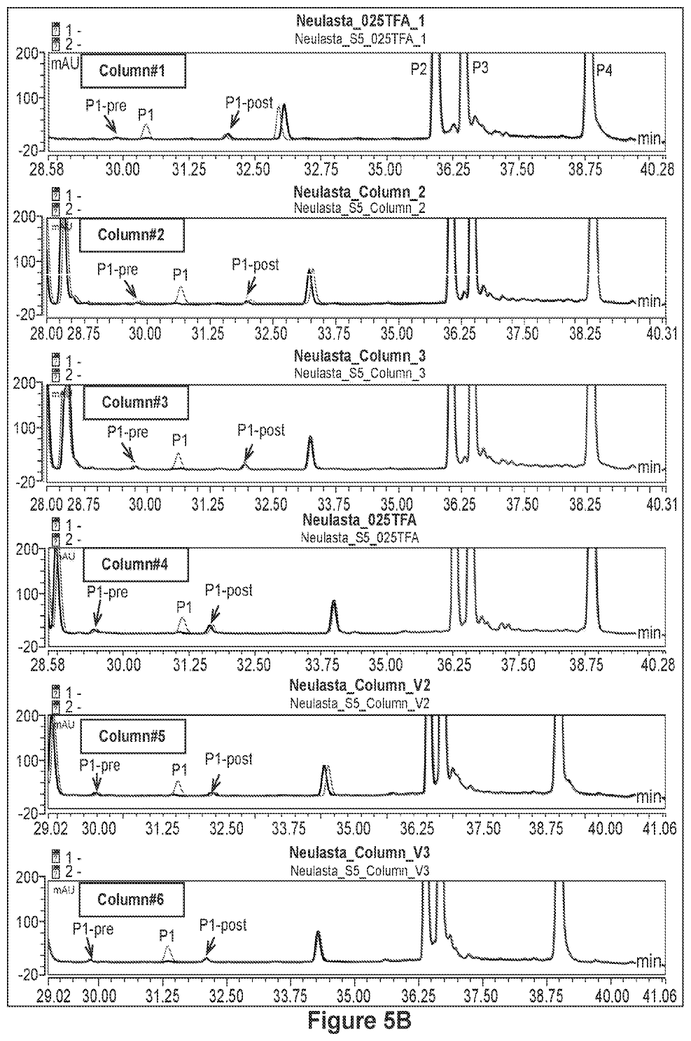

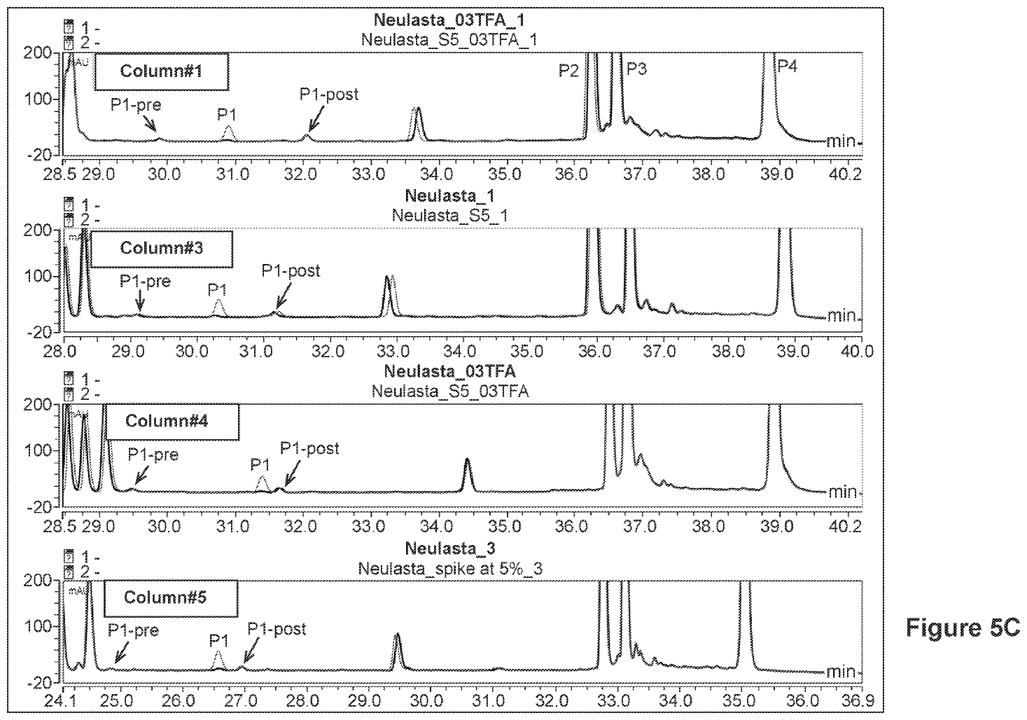

FIG. 5A-5C shows the peptide map profiles near the peptide of interest, six different UPLC column lots at 3 different TFA concentrations (0.02, 0.025 and 0.03% v/v) were tested. The peptide identifications of the labeled peaks are shown in Table 2.

TABLE 2
Peptides identifications for labeled peaks in chromatographic
profiles in FIG. 5A-5C.

| | | Mass (monoisotopic) | |
|---|---|---|---|
| Label | Identified Peptide | Theoretical | Determined |
| P1-pre | Q91-T106 (Q91 + NH3 loss) | 1622.80 | 1622.81 |
| P1 | M1-L15 | 1544.79 | 1544.79 |
| P1-post | Q91-L104 (Q91 + NH3 loss) | 1406.72 | 1406.73 |
| P2 | L51-L76 | 2616.33 | 2616.32 |
| P3 | L51-L79 | 2944.50 | 2944.49 |
| P4 | Peg-M1-L15 | — | — |

It is apparent from FIG. 5A-5C that the separation of peptide M1-L15 from nearby peptides (P1-pre and P1-post) depends on the column lot being used. Fortunately, separation of these three peaks can be optimized by varying the TFA concentration in the mobile phase. In the case when a specific column lot causes co-elution of two peaks, TFA concentration can be varied from 0.02% (v/v) to 0.03% (v/v) to achieve separation, as exemplified in FIG. 5A-5C. A TFA concentration of 0.025% (v/v) was selected because all six column lots achieved well resolved P1 peak with 0.025% (v/v) TFA compared to the other two concentrations.

### EXAMPLE 2

### Method Qualification

A. Specificity and Carryover

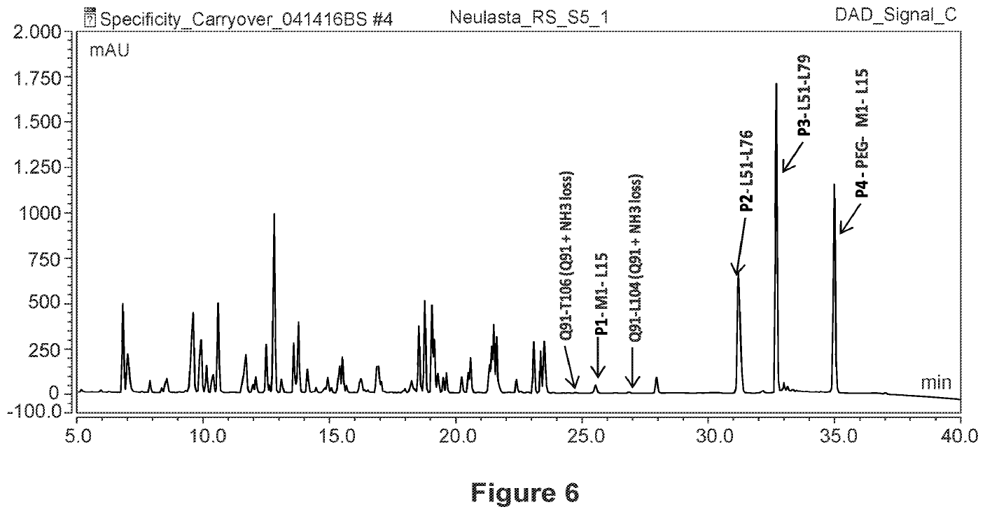

To establish specificity, the major reference peaks (P2, P3, and P4) as well as a few nearby peaks of interest were first identified through online mass spectrometric (MS) detection (FIG. 6). No distinct mass can be determined from mass spectrum of peak P4, presumably due to the heterogeneous nature of the mPEG-aldehyde. As described in section 5.1.3, peak P4 was confirmed to be PEG-M1-L15 by N-terminal sequencing.

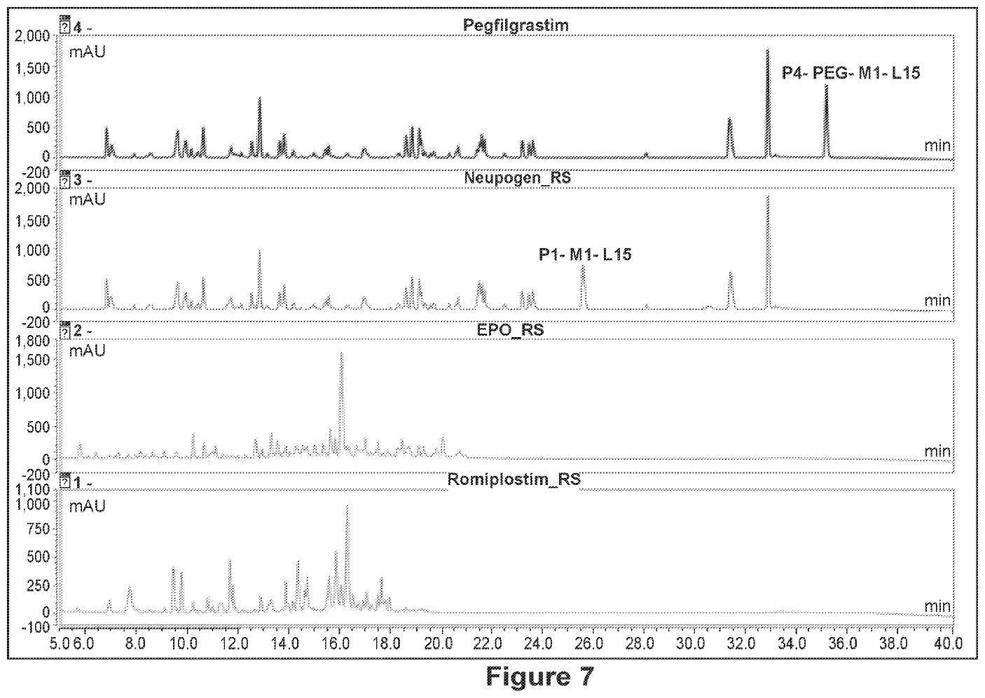

To establish that the test method is specific to product-related components, a unique chromatographic profile must be achieved for Pegfilgrastim drug substance (DS) after digested with pepsin. Products that are similar in size or manufactured at the same site including Neupogen® (filgrastim), Epogen® [Epoetin alfa, (EPO)] and Nplate® (Romiplostim) were analyzed in parallel with Pegfilgrastim and the resulting chromatograms are shown in FIG. 7. These chromatograms are clearly different, establishing the specificity of the assay to distinguish these products. Among these products, Neupogen differs from Pegfilgrastim only by N-terminal PEGylation, which can be clearly distinguished by the absence of reference peak P4 (PEGylated N-terminal peptide) and the appearance of the peak P1 (free N-terminal peptide) in Neupogen pepsin digest.

To assess carryover of the method, an enzyme blank was injected after injection of digested Pegfilgrastim RS spiked with 5% Filgrastim RS. Carryover was calculated based on the relative percent peak areas determined for each reference peak in the blank run compared to spiked Pegfilgrastim RS sample. As no peak was detected in the region of retention times in both pre as well as post blank run, carryovers for all these peaks are 0%.

B. Linearity

The linearity experiment was designed to determine the assay ability (within a given range) to obtain test results that are directly proportional to the percent level of free N-terminal impurity. To establish the linearity of the method, as seen in Table 3, Pegfilgrastim RS spiked with Filgrastim RS at 0.5%, 1.0%, 1.5%, 2.0% and 2.5% levels (to make the final free N-terminal methionine of approximately 1.0%, 1.5%, 2.0%, 2.5% and 3.0%, respectively) were digested and analyzed in triplicates (Table 4). Together with the un-spiked Pegfilgrastim RS analysis (two values per each linearity run), there are a total of 6 levels for linearity assessment.

TABLE 3
Sample preparation for the linearity experiment.

| | Pegfilgrastim | Filgrastim | % | Estimated | Theoretical |
|---|---|---|---|---|---|
| | RS (0.97 | RS (0.97 | Spiked | % Free N- | Con- |
| | mg/mL) | mg/mL) | Filgrastim | terminal | centration |
| Level | volume (μL)* | volume (μL)* | RS | Methionine | (mg/mL) |
| 1 | 1990 | 10 | 0.5 | 1.0 | 0.97 |
| 2 | 1980 | 20 | 1.0 | 1.5 | 0.97 |
| 3 | 9850 | 150 | 1.5 | 2.0 (Target) | 0.97 |
| 4 | 1960 | 40 | 2.0 | 2.5 | 0.97 |
| 5 | 1950 | 50 | 2.5 | 3.0 | 0.97 |

TABLE 4
Determined % free N-terminal methionine at each level and their average and % RSD.

| | Determined % Free N-terminal Methionine | | | | | |
|---|---|---|---|---|---|---|
| | Level 0 | Level 1 | Level 2 | Level 3 | Level 4 | Level 5 |
| Run# | Unspike | 0.5% spike | 1.0% spike | 1.5% spike | 2.0% spike | 2.5% spike |
| 1 | 0.498 | 0.975 | 1.443 | 1.973 | 2.504 | 2.905 |
| 2 | 0.452 | 0.986 | 1.485 | 1.942 | 2.474 | 2.952 |
| 3 | 0.473 | 0.964 | 1.409 | 1.932 | 2.466 | 3.029 |
| 4 | 0.477 | — | — | — | — | — |
| 5 | 0.462 | — | — | — | — | — |
| 6 | 0.462 | — | — | — | — | — |
| Average | 0.471 | 0.975 | 1.446 | 1.949 | 2.481 | 2.962 |
| Std dev | 0.016 | 0.011 | 0.038 | 0.021 | 0.020 | 0.063 |
| % RSD | 3.41 | 1.13 | 2.63 | 1.10 | 0.81 | 2.11 |

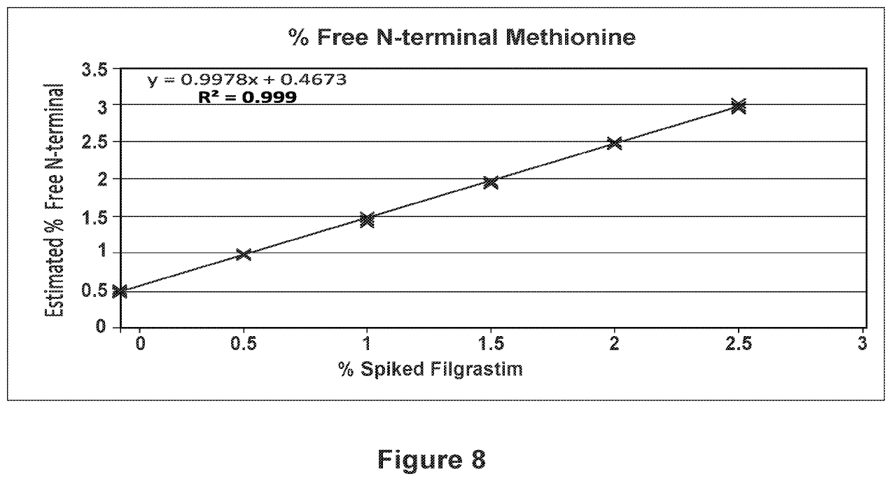

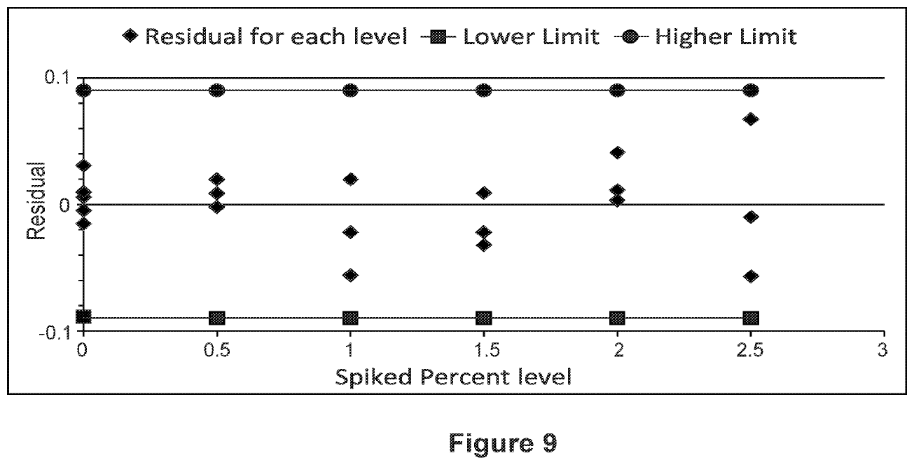

The determined levels of % free N-terminal methionine were plotted against the spike level in FIG. 8. Linear regression is performed to obtain slope, y-intercept and R² values. Also shown in FIG. 9 is the residual plot, from which residual sum of squares and residual standard deviation are calculated. Table 5 shows these determined values for the linearity of the measurement in the range studied.

TABLE 5
Regression analysis values for linearity.

| Parameter | Determined value |
|---|---|
| The coefficient of | 0.999 |
| determination (R²) | |
| Residual plot | See FIG. 9 |
| | Maximum residual value = 0.067 |
| Residual Standard | 0.029% |
| Deviation | |
| Range | 0.5%-3% |

C. Precision (Repeatability/Intermediate Precision)

Repeatability and intermediate precision were evaluated for both peptide map profile and free N-terminal methionine quantitation. To evaluate repeatability and intermediate precision for peptide map profile, a total of six runs, with 4 sample injections and 4 blank injections in each run, were performed on 4 different HPLC systems, by 2 different analysts in 2 different labs and using 3 different columns. Total area under the curve (tAUC), the peak areas (pAUC) and retention times (RT) ratios of the reference peaks (P2/P4 and P3/P4) for each sample injection, peak-to-peak noise of each enzyme-only blank injection as well as retention time difference of bracketing Filgrastim spiked reference standard were recorded and shown in Table 6. The highest % CV for retention time and peak area parameters per run is less than 0.3% and 7%, respectively. These results demonstrate good repeatability of the peptide map profile.

TABLE 6
Experiments performed for testing intermediate precision for peptide mapping

| Run 1 | Run 2 | Run 3 | Run 4 | Run 5 | Run 6 | |
|---|---|---|---|---|---|---|
| Analyst (site) | | | | | | |
| 1 (ATO AS) | 1 (ATO AS) | 2 (AML AS) | 2 (AML AS) | 1 (ATO AS) | 1 (ATO AS) | |
| Equipment | | | | | | |
| 652940 | 878875 | HPLC 30 | HPLC 32 | 652940 | 878875 | For all the values |
| Column Lot | | | | | | of each |
| 0124 | 0133 | 0127 | 0124 | 0133 | 0127 | parameter |

Total Area

| | | | | | | | | |
|---|---|---|---|---|---|---|---|---|
| 1 | 1041.3 | 1199.9 | 1216.3 | 1138.1 | 1111.9 | 1124.7 | | |
| 2 | 1134.4 | 1226.4 | 1212.8 | 1165.4 | 1115.7 | 1107.6 | | |
| 3 | 1125.3 | 1217.3 | 1217.0 | 1161.8 | 1119.8 | 1107.8 | Average | 1155.8 |
| 4 | 1132.8 | 1221.3 | 1214.2 | 1160.7 | 1127.4 | 1139.1 | StdDev | 49.375 |
| Average/run | 1108.4 | 1216.2 | 1215.1 | 1156.5 | 1118.7 | 1119.8 | | |
| StdDev/run | 44.928 | 11.503 | 1.937 | 12.396 | 6.609 | 15.158 | | |
| % CV | 4.053 | 0.946 | 0.159 | 1.072 | 0.591 | 1.354 | | |

Ratio P2/P4 (RT)

| | | | | | | | | |
|---|---|---|---|---|---|---|---|---|
| 1 | 0.930 | 0.897 | 0.926 | 0.929 | 0.924 | 0.924 | | |
| 2 | 0.930 | 0.895 | 0.926 | 0.929 | 0.924 | 0.923 | | |
| 3 | 0.930 | 0.894 | 0.926 | 0.929 | 0.924 | 0.923 | Average | 0.921 |
| 4 | 0.930 | 0.892 | 0.926 | 0.929 | 0.924 | 0.921 | StdDev | 0.012 |
| Average/run | 0.930 | 0.895 | 0.926 | 0.929 | 0.924 | 0.923 | | |
| StdDev/run | 0.000 | 0.002 | 0.000 | 0.000 | 0.000 | 0.001 | | |
| % CV | 0.000 | 0.233 | 0.013 | 0.034 | 0.000 | 0.136 | | |

Ratio P2/P4 (Area)

| | | | | | | | | |
|---|---|---|---|---|---|---|---|---|
| 1 | 1.184 | 1.064 | 0.833 | 0.935 | 0.984 | 1.029 | | |
| 2 | 1.087 | 1.040 | 0.834 | 0.926 | 0.985 | 1.065 | | |
| 3 | 1.090 | 1.041 | 0.838 | 0.932 | 1.015 | 1.071 | Average | 0.994 |
| 4 | 1.010 | 1.033 | 0.835 | 0.964 | 1.009 | 1.058 | StdDev | 0.0923 |
| Average/run | 1.093 | 1.045 | 0.835 | 0.939 | 0.998 | 1.056 | | |
| StdDev/run | 0.071 | 0.013 | 0.003 | 0.017 | 0.016 | 0.019 | | |
| % CV | 6.517 | 1.290 | 0.299 | 1.810 | 1.610 | 1.763 | | |

Ratio P3/P4 (RT)

| | | | | | | | | |
|---|---|---|---|---|---|---|---|---|
| 1 | 0.939 | 0.937 | 0.931 | 0.934 | 0.937 | 0.941 | | |
| 2 | 0.939 | 0.937 | 0.931 | 0.934 | 0.937 | 0.941 | | |
| 3 | 0.938 | 0.936 | 0.931 | 0.934 | 0.937 | 0.941 | Average | 0.936 |
| 4 | 0.938 | 0.936 | 0.931 | 0.934 | 0.938 | 0.941 | StdDev | 0.0032 |
| Average/run | 0.939 | 0.937 | 0.931 | 0.934 | 0.937 | 0.941 | | |
| StdDev/run | 0.001 | 0.001 | 0.000 | 0.000 | 0.000 | 0.000 | | |
| % CV | 0.062 | 0.062 | 0.013 | 0.026 | 0.053 | 0.000 | | |

Ratio P3/P4 (Area)

| | | | | | | | | |
|---|---|---|---|---|---|---|---|---|
| 1 | 0.923 | 0.982 | 1.325 | 1.048 | 1.120 | 0.996 | | |
| 2 | 1.039 | 0.979 | 1.295 | 1.048 | 1.133 | 0.963 | | |
| 3 | 1.037 | 0.990 | 1.306 | 1.041 | 1.128 | 0.974 | Average | 1.074 |
| 4 | 1.041 | 0.984 | 1.319 | 0.988 | 1.125 | 0.990 | StdDev | 0.1219 |
| Average/run | 1.010 | 0.984 | 1.311 | 1.031 | 1.127 | 0.981 | | |
| StdDev/run | 0.058 | 0.005 | 0.014 | 0.029 | 0.005 | 0.015 | | |
| % CV | 5.745 | 0.472 | 1.045 | 2.830 | 0.484 | 1.534 | | |

Noise (Pto P)

| | | | | | | | | |
|---|---|---|---|---|---|---|---|---|
| 1 | 0.417 | 0.505 | 0.091 | 0.113 | 0.367 | 0.388 | | |
| 2 | 0.377 | 0.410 | 0.105 | 0.088 | 0.292 | 0.480 | | |
| 3 | 0.366 | 0.469 | 0.044 | 0.091 | 0.386 | 0.530 | Average | 0.308 |
| 4 | 0.377 | 0.483 | 0.064 | 0.096 | 0.352 | 0.503 | StdDev | 0.170 |

RT for spiked RS

| | | | | | | | | |
|---|---|---|---|---|---|---|---|---|
| Begin | 31.150 | 25.631 | 32.245 | 31.858 | 30.263 | 26.659 | | |
| End | 31.022 | 25.538 | 32.219 | 31.792 | 30.307 | 26.571 | Average | 0.06 |
| Difference | 0.128 | 0.093 | 0.026 | 0.066 | −0.044 | 0.088 | StdDev | 0.061 |

To evaluate repeatability and intermediate precision for free N-terminal methionine quantitation, a total of four runs were performed on the Pegfilgrastim sample spiked with 1.5% Filgrastim, with triplicate analyses in each run, on 4 different HPLC systems, by 2 different analysts in 2 different labs and using 3 different columns. Table 7 shows the results of these analyses, for repeatability standard deviation and intermediate precision standard deviation

TABLE 7
Intermediate precision data for N-terminal methionine quantitation

| Lab | ATO | ATO | AML | AML |
|---|---|---|---|---|
| Experiments | Linearity | Precision 2 | Precision 3 | Precision 4 |
| Equipment | 652940 | 878875 | HPLC 30 | HPLC 32 |
| Column Lot | 0124 | 0133 | 0127 | 0124 |
| | % Free N-terminal Methionine | | | |
| Replicate 1 | 1.973 | 2.022 | 1.985 | 1.898 |
| Replicate 2 | 1.942 | 1.949 | 1.995 | 1.925 |
| Replicate 3 | 1.932 | 2.004 | 2.009 | 1.922 |

ANOVA

| Source of Variation | SS | df | MS | F | P-value | F crit |
|---|---|---|---|---|---|---|
| Between Groups | 0.0133 | 3 | 0.0044 | 7.8193 | 0.0092 | 4.0662 |
| Within Groups | 0.0045 | 8 | 0.0006 | | | |
| Total | 0.0178 | 11 | | | | |

| | Determined value |
|---|---|
| Repeatibility standard deviation | 0.0238 |
| Intermediate precision standard deviation | 0.0431 |

D. Accuracy

Data collected in the linearity experiment were used to evaluate the accuracy of the method in measuring the amount of free N-terminal methionine. To get an accurate theoretical amount of free N-terminal methionine in each sample, the small amount of N-terminal methionine in the un-spiked Pegfilgrastim RS was first determined from the six system suitability runs during the linearity assessment (determined to be an average of 0.471% as shown in Table 8).

TABLE 8
Free N-terminal methionine determination in the un-spiked Pegfilgrastim RS.

| | % Free N-Terminal Methionine Determination | | | | | | |
|---|---|---|---|---|---|---|---|
| Sample | 1 | 2 | 3 | 4 | 5 | 6 | Average |
| Level 0 | 0.498 | 0.452 | 0.473 | 0.477 | 0.462 | 0.462 | 0.471 |

Due to the low level of free N-terminal methionine in un-spiked Pegfilgrastim RS, the measurement error is negligible for calculating theoretical concentration of spiked samples. The theoretical level of free N-terminal methionine can be calculated based on the volume of spiked Filgrastim (VF) and the volume of un-spiked Pegfilgrastim RS (VPF) from the following formula.

(0.471% VPF+100% VF)/(VPF+VF)

Table 9 compares the theoretical and experimentally determined % free N-terminal methionine of samples at different levels of free N-terminal methionine. Accuracy was calculated from the average determined values of the triplicate analysis and represented as a percentage in Table 9. Accuracy of measurement was found to be acceptable for the 1.5%-spike level and all other measured concentrations.

TABLE 9
Accuracy of free N-terminal methionine quantitation at different concentrations.

| % Free N- | Free N-terminal % | | |
|---|---|---|---|
| Methionine | Expected | | |
| terminal | Theoretical | Determined | % Accuracy |
| Original | — | 0.471 | — |
| 1.00% | 0.968 | 0.975 | 100.7% |
| 1.50% | 1.466 | 1.446 | 98.6% |
| 2.00% | 1.964 | 1.949 | 99.2% |
| 2.50% | 2.461 | 2.481 | 100.8% |
| 3.00% | 2.959 | 2.962 | 100.1% |

E. Range

In the range of 1.0-3.0% free N-terminal methionine level, data presented in this report demonstrated acceptable linearity, accuracy and precision, demonstrating the method is capable of determining % free N-terminal methionine in this range.

F. Limit of Detection (LOD) and Limit of Quantitation (LOQ)

In one embodiment described herein, the method protocol requires running two Pegfilgrastim RS injections and two injections of Pegfilgrastim RS spiked with 5% Filgrastim RS in each sequence, facilitating determination of % N-terminal methionine in Pegfilgrastim RS. The linearity assessment is consisted of three sequences, generating 6 measurements of % free N-terminal methionine in Pegfilgrastim RS. Additionally, three more runs for intermediate precision evaluation generated 6 more measurements. The standard deviation of the 12 measurements was calculated and used to determine LOD and LOQ with the following equations. The results are shown in Table 10.

LOD=3.3σ

LOQ=10σ

TABLE 10
LOD and LOQ for N-terminal methionine determination.

| Experiments | % Free N-terminal Methionone in Pegfilgrastim RS | | | | | |
|---|---|---|---|---|---|---|
| From Linearity | 0.498 | 0.452 | 0.473 | 0.477 | 0.462 | 0.462 |
| From Precison _AML | 0.477 | 0.473 | 0.490 | 0.499 | — | — |
| From Precision_ATO | 0.533 | 0.512 | — | — | — | — |

| Standard Deviation (σ) | 0.0233 |
|---|---|
| | Determined Value |
| LOD (3.3 * σ) | 0.08% |
| LOQ (10 * σ) | 0.23% |

G. Sample Stability After Preparation

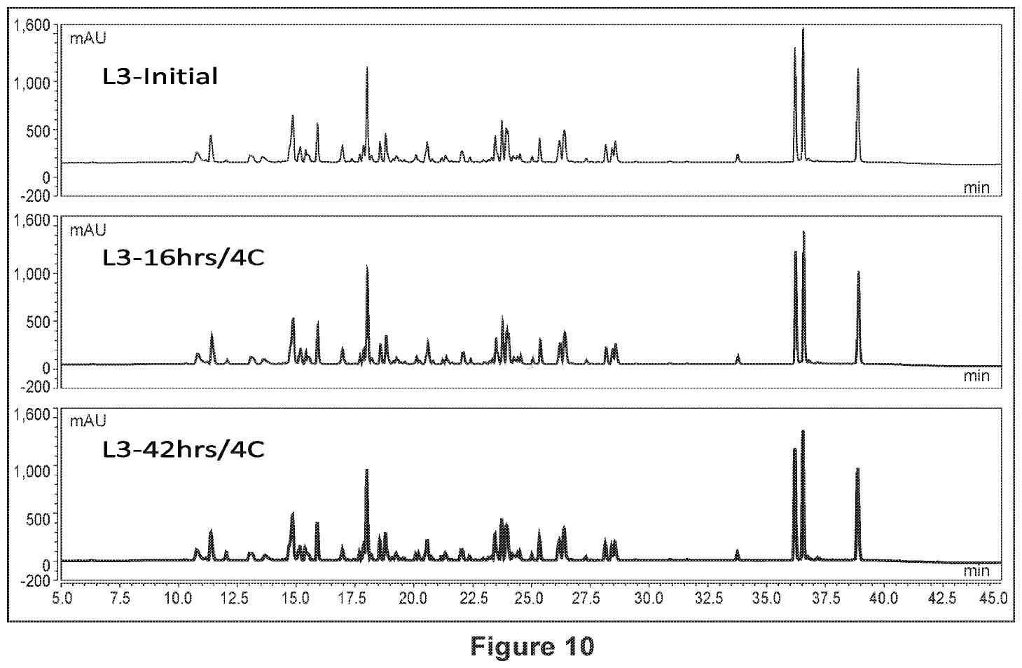

To demonstrate the stability of the digested samples in the cooled auto-injector before analysis, two digested samples were injected before and after a long sequence (16 hours interval), as well as on the second day (42 hour interval). The resulting chromatograms yield no significant difference (FIG. 10). The determined difference in % free N-terminal methionine of sample at 16 hrs at 4° C. as well as 42 hrs at 4° C. is less than 0.4% (Table 11) suggesting sample is stable up to 42 hrs at 4° C.

TABLE 11
Level 3 sample stability in auto-injector over 42 hours at 4° C.

| Linearity | % Free N-terminal Methionine Determination | | | | |
|---|---|---|---|---|---|
| ex- | Level 3 | L3-16 hrs at 4° C. | | L3-42 hrs at 4° C. | |
| per- | (L3) | | Differ- | | Differ- |
| iment | Initial | Determined | ence | Determined | ence |
| 1 | 1.973 | 1.940 | 0.033 | 1.906 | 0.067 |
| 2 | 1.942 | 1.900 | 0.042 | 1.876 | 0.066 |
| 3 | 1.932 | 1.896 | 0.036 | — | — |

### EXAMPLE 3

### Robustness

A seven-factor designed experiment (DOE) was performed to evaluate the robustness of the method (Table 12). The HPLC column and enzyme lot factors are categorical and a matrix was created in Minitab® 15.1.30.0 statistical software. One analysis per run of a control sample (Pegfilgrastim RS spiked with 1.5% Filgrastim RS) was performed.

TABLE 12
Experimental Design for Robustness Evaluation.

| | Parameter | | | | | | |
|---|---|---|---|---|---|---|---|
| | Column | | Digestion | Digestion | Digestion | | HPLC |
| | Temperature ± | % TFA ± | puffer | Time ± | Temp ± | Enzyme | Column |
| Run | 2° C. | 12% | pH ± 0.1 | 10% | 2° C. | Lot (E)* | Lot (L)† |
| 1 | 52 | 0.022 | 2.3 | 13.5 | 35 | E2 | L1 |
| 2 | 50 | 0.025 | 2.2 | 15 | 37 | E1 | L1 |
| 3 | 52 | 0.022 | 2.1 | 16.5 | 39 | E1 | L1 |
| 4 | 48 | 0.022 | 2.1 | 13.5 | 39 | E2 | L2 |
| 5 | 50 | 0.025 | 2.2 | 15 | 37 | E2 | L1 |
| 6 | 50 | 0.025 | 2.2 | 15 | 37 | E1 | L2 |
| 7 | 52 | 0.028 | 2.3 | 16.5 | 39 | E2 | L2 |
| 8 | 52 | 0.028 | 2.1 | 13.5 | 35 | E1 | L2 |
| 9 | 50 | 0.025 | 2.2 | 15 | 37 | E2 | L2 |
| 10 | 48 | 0.028 | 2.1 | 16.5 | 35 | E2 | L1 |
| 11 | 48 | 0.028 | 2.3 | 13.5 | 39 | E1 | L1 |
| 12 | 48 | 0.022 | 2.3 | 16.5 | 35 | E1 | L2 |

*E1 - Sigma part # P6887 lot 074K7717 and E2- Sigma part # P6887 lot SLBM3033V;
†L1 - Column lot 0124(S/N01243535515703), L2- Column Lot 0133(S/N01333532416856)

To evaluate the method robustness regarding the peptide map profile, the average values of total peak area, peak to peak noise (p to p), P2/P4 and P3/P4 ratio for retention time as well as peak area from the center point experiments are calculated. Acceptable range for each parameter was established using the following equation.

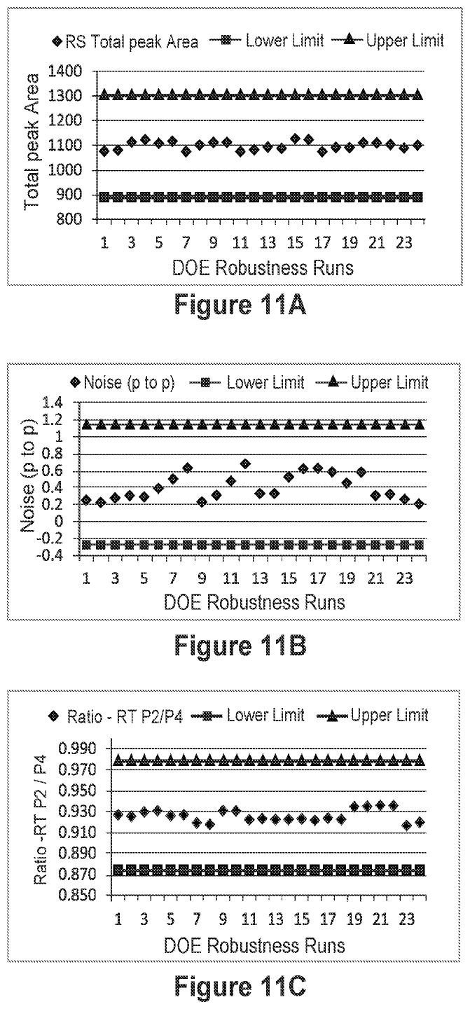

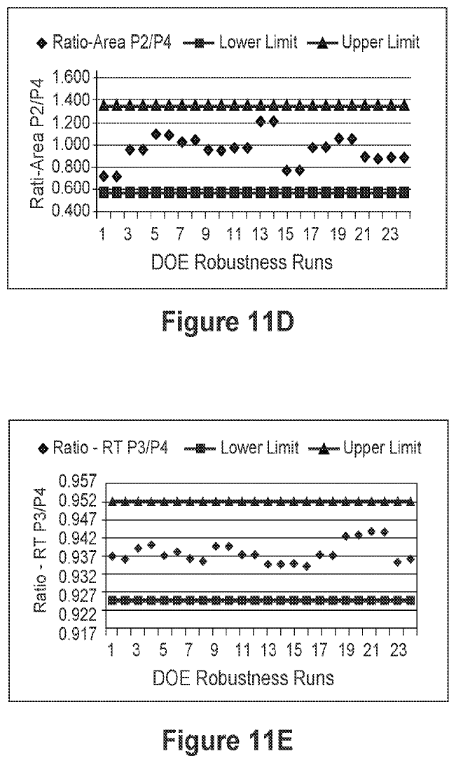

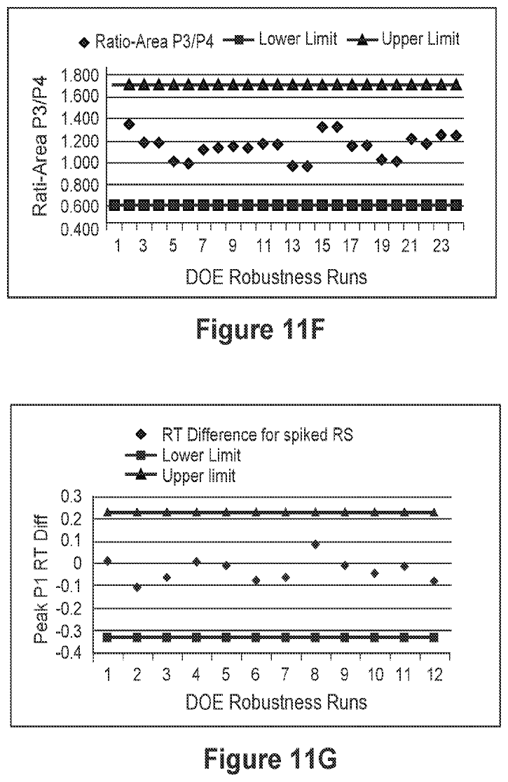

Acceptable range=average of centerpoint experiments±3*(StdDev)*(Mn)

Where Mn=1.403 is the multiplier for intermediate precision measurement (n=24) (Hahn and Meeker, 2011), and StdDev are the standard deviation values obtained from the intermediate precision experiment. As seen in FIG. 11A-11G, total peak area, Noise (p to p), peak areas and retention times for four reference peaks are all within the acceptance range.

To evaluate the method robustness for free N-terminal methionine determination, the average value of free N-terminal methionine determined from the center point experiments is calculated. Acceptable range for other experiments is calculated from the following equation

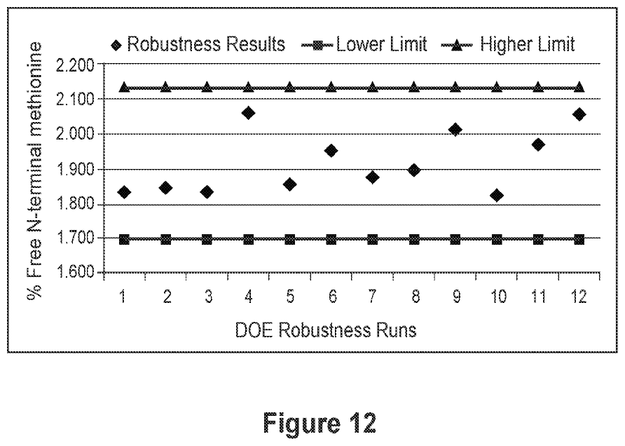

Acceptable range=average of centerpoint experiments±3*(Intermediate precision standard deviation)*(Mn)

Where Mn=1.698 is the multiplier for intermediate precision measurement (n=12) (Hahn, Gerald J., and William Q. Meeker. Statistical intervals: a guide for practitioners. Vol. 92. John Wiley & Sons, 2011). As seen in FIG. 12, determined % free N-terminal methionine are within acceptable range for all the experiments, demonstrating the robustness of the method in determining the level of N-terminal free methionine.

### EXAMPLE 4

### Exemplary Protocol—Materials and Methods

The following materials and methods provide an exemplary method, as further disclosed in the above examples, to confirm the identity of Pegfilgrastim, and to determine the level of % free N-terminal methionine with excellent specificity, precision, accuracy, linearity, LOD, LOQ, range and robustness.

Pegfilgrastim is digested with pepsin under acidic condition. Digested peptides are separated by reversed phase chromatography and detected by UV at 214 nm. Percent free N-terminal methionine (without PEG) is determined by spiking known amount (5%) of Filgrastim in the Pegfilgrastim sample. Pepsin peptide map profile of sample is compared with Pegfilgrastim reference standard to confirm identity.

To perform pepsin digestion of Filgrastim or Pegfilgrastim, (spiked as well as regular sample), 120 μL of protein sample at 1 mg/mL with 60 μL of 0.3M phosphate buffer at pH 2.2 and 10 μL of pepsin solution at 0.48 mg/mL, mixed well and incubated at 37° C. for 15 minutes. After incubation, the digestion was quenched by adding 10 μL of 1N NaOH.

15 ug of digested sample peptides were separated on Waters CSH reversed phase column (2.1×100 mm) at 50° C. with a 40-minute acetonitrile gradient (2 to 25% in 19 minutes followed by 25 to 30% in 10 minutes then 30 to 99% in 10 minutes followed by column washing and re-equilibration) with 0.025% TFA. Flow rate was maintained at 0.2 mL/minute, UV detection was achieved at 214 nm.

Percent free N-terminal determination was done by using free N-terminal peptide peak area of spiked sample (at 5%) and unspiked sample using following formula

$$\% Free N - terminus of Pegfilgrastim sample = \frac{5 ( A 0 )}{( A 1 ) - 0.95 * ( A 0 )}$$

A0=Peak area of free N-terminal peptide detected in pegfilgrastim sample
A1=Peak area of free N-terminal peptide detected in the pegfilgrastim sample spiked with 5% filgrastim

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of measuring the amount of unmodified N-terminus of a human granulocyte colony-stimulating factor (G-CSF) polypeptide, comprising the steps of:
(a) incubating a sample comprising the G-CSF polypeptide with a non-specific protease under conditions that allow cleavage at one or more sites within the G-CSF polypeptide and only once between N-terminal methionine at position 1 and Lysine at position 16;
(b) separating the cleavage products generated in step (a); and
(c) measuring the amount of unmodified, free N-terminus of the G-CSF polypeptide by comparing to a control standard.

2. The method of claim 1 wherein the G-CSF polypeptide is recombinant.

3. The method of claim 1 wherein said sample comprises a mixture of modified G-CSF polypeptide and unmodified G-CSF polypeptide, and wherein the modified G-CSF polypeptide comprises at least one polyethylene glycol (PEG) modification.

4. The method of claim 1 wherein the G-CSF polypeptide is selected from the group consisting of Pegfilgrastim (Neulasta®), Pegfilgrastim-jmdb (Fulphila®), INN-Pegfilgrastim (Pelgraze®), Lapelga®, Pelmeg®, Pegfilgrastim-cbqv (Udenyca®), Pegfilgrastim-bmez (Ziextenzo®), and Grasustek®.

5. The method of claim 4 wherein the G-CSF polypeptide is Pegfilgrastim (Neulasta®).

6. The method of claim 1 wherein the non-specific protease cleaves between leucine at position 15 and leucine at position 16 and produces a peptide of 15 amino acids in length (peptide M1-L15).

7. The method of claim 1 wherein the non-specific protease is pepsin.

8. The method of claim 1 wherein the conditions in step (a) comprise incubating (a) at a pH of about 1.5 to about 4.0, (b) at a temperature of about 25° C. to about 60° C., and (c) for a time of about 5 minutes to about 60 minutes.

9. The method of claim 8 wherein the conditions comprise incubating (a) at a pH of about 2.2, (b) at a temperature of about 37° C., and (c) for a time of about 15 minutes.

10. The method of claim 1 wherein the separation of step (b) is carried out under conditions that allow separation of peptide M1-L15 from other cleavage products.

11. The method of claim 10 wherein the separation of step (b) is carried out by a method selected from chromatography and electrophoresis.

12. The method of claim 11 wherein the chromatography is selected from the group consisting of high-performance liquid chromatography (HPLC) and ultrahigh-performance liquid chromatography (UHPLC).

13. The method of claim 10 wherein the HPLC is reversed phase HPLC (RP-HPLC).

14. The method of claim 13 wherein the chromatography comprises a column and trifluoroacetic acid (TFA) at a concentration of about 0.01% v/v to about 0.2% v/v.

15. The method of claim 14 wherein the TFA concentration is about 0.02% v/v to about 0.03% v/v.

16. The method of claim 15 wherein the TFA concentration is about 0.025% v/v.

17. The method of claim 1 wherein the measuring step (c) is carried out by mass spectrometry.

18. The method of claim 17 wherein the mass spectrometry is selected from electrospray MS and Matrix-assisted laser desorption ionization-time of flight mass spectrometry (MALDI-TOF MS).

19. The method of claim 1 wherein the control standard comprises a known amount of modified G-CSF polypeptide and a known amount of unmodified G-CSF polypeptide.

20. A method of measuring the amount of unPEGylated, free N-terminus of Pegfilgrastim (Neulasta®) comprising the steps of:
(a) incubating a sample comprising Pegfilgrastim (Neulasta®) with a non-specific protease under conditions that allow cleavage at one or more sites within the Pegfilgrastim (Neulasta®) and only once between N-terminal methionine at position 1 and Lysine at position 16, and wherein said conditions comprise incubating (a) at a pH of about 2.2, (b) at a temperature of about 37° C., and (c) for a time of about 15 minutes;
(b) separating the cleavage products generated in step (a) by reversed phase HPLC (RP-HPLC); and
(c) measuring the amount of unPEGylated, free N-terminus of Pegfilgrastim (Neulasta®) by comparing to a control standard.
